(19) United States
(12) Patent Application Publication
(10) Pub. No.: US 2011/0189119 A1
(43) Pub. Date: Aug. 4, 2011

(54) COMPOSITION FOR PERMANENT WAVE

(76) Inventor: Jae Hwa Jin, Seoul (KR)

(21) Appl. No.: 12/452,631

(22) PCT Filed: Jun. 5, 2009
(86) PCT No.: PCT/KR2009/003001

§ 371 Date: Apr. 20, 2011

(51) Int. Cl. A61K 8/97; A61K 8/64; A61K 8/67; A61Q 5/04; A61Q 7/00

(52) U.S. Cl. 424/70.51

(57) ABSTRACT

Disclosed is a low-irritant, high-performance composition for permanent waves. It comprises L-cysteine or a mixture of L-cysteine and L-acetylcysteine as a reducing agent, for breaking disulfide bridges of cystines to form cysteines; L-arginine for swelling keratin fibers to facilitate deep invasion of the reducing agent into the keratin fibers; and a citrus peel powder or extract, containing peptides for promoting growth of dermal papilla cells and D-limonene for preventing depilation, and optionally with histidine for regenerating the hair, a powder or extract of green tea leaves for exerting an astringent effect on a scalp, chlorella for exerting an anti-bacterial and deodorizing effect on the scalp, a powder or extract of cactus for preventing a horny tissue from being generated on the scalp or desquamating upper layers of dead skin cells from the scalp, and polyquaternium or Hydroxyethylcellulose as a viscosity-inducing substance, whereby the human hair can be healthily changed from waves to straight or vice versa within a short time without irritation to the skin and the scalp and the formed curls can be prolonged with elasticity.

### TECHNICAL FIELD

The present invention relates to a low-irritant, high-performance composition for permanent hair waves. More particularly, the present invention relates to a composition for permanent waves. It comprises L-cysteine or a mixture of L-cysteine and L-acetylcysteine as a reducing agent, for breaking disulfide bridges of cystines to form cysteines; L-arginine for swelling keratin fibers to facilitate deep invasion of the reducing agent into the keratin fibers; and a citrus peel powder or extract, containing peptides for promoting growth of dermal papilla cells and D-limonene for preventing depilation, and optionally with histidine for regenerating the hair, a powder or extract of green tea leaves for exerting an astringent effect on a scalp, chlorella for exerting an anti-bacterial and deodorizing effect on the scalp, a powder or extract of cactus for preventing a horny tissue from being generated on the scalp or desquamating upper layers of dead skin cells from the scalp, and polyquaternium or Hydroxyethylcellulose as a viscosity-inducing substance, whereby the human hair can be healthily changed from waves to straight or vice versa within a short time without irritation to the skin and the scalp and the formed curls can be prolonged with elasticity.

### BACKGROUND ART

Keratins, which are the main constituent of structures that grow from the skin, such as hairs, are fibrous structural proteins composed of 18 different amino acids, featuring a high content of crosslinking by disulfide bridges (one for every 10˜20 amino acid residues), which play a crucial role in the folding and stability of some proteins and thus in determining their tertiary structure. The disulfide bridge results from the formation of cystine, represented by the chemical formula HO₂CC(NH₂)HCH₂S—SCH₂C(NH₂)HCO₂H, by the oxidation of two cysteine residues.

Typically, a permanent waving formula is composed of a first formulation based on a reducing agent and a second formulation based on oxidizing agent (neutralizer).

In the mechanism of permanent waves, the first formulation (reducing agent) is applied to hairs to break open the disulfide bridges of cystine [HO₂CC(NH₂)HCH₂S—SCH₂C(NH2)HCO₂H] and form cysteine [HO₂CC(NH₂)HCH₂SH] (the reduction step) prior to, subsequent to or concurrent with the application of physical or mechanical force to the hairs using rollers or rods to shape the hairs into the desired style, such as waves, curls or straight (tensile force step), followed by oxidizing cysteine with the second formulation into cystine to fix the desired style of the hair (the oxidation or fixing step).

The new shape of the hair established by the above-mentioned chemical treatment remains unchanged even though the hair undergoes washing with water or shampooing, while upon simple non-permanent treatment such as hair setting, applying water or shampoo causes the established hair shape to change. Thus, a permanent wave, commonly called a perm, is chemically and/or thermally treating hair to deform straight hair into waves or curls and vice versa.

The first formulation based on a reducing agent is typically in the form of viscous formulas such as lotions, concentrates, creams, gels, etc. Typical examples of the reducing agent include cysteamine and its derivatives, thiolactic acid, thioglycolic acid and its esters, particularly, glycerol monothioglycolate, thioglycerol, mercaptopropionic acid, etc. An anionic, cationic, amphoteric or non-ionic surfactant may be used as an additive in the first formulation. Typically, the first formulation comprises other additional components, such as hair protecting agents, dandruff suppressors, thickeners, suspending agents, sequestering agents, opacifiers, dye, UV protectors, fragrance, preservatives, etc, with ethanol, propanol, isopropanol or glycerol serving as a solvent.

On the other hand, the oxidizing agent in the second formulation is typically a diluted bromic acid or hydrogen peroxide solution (3˜6% aqueous solution).

Such conventional permanent wave formulas suffer from the following disadvantages.

The typical reducing agents thioglycolic acid and esters thereof show high reducing ability whereas the mercaptan compounds and their corresponding derivatives are difficult to control. One disadvantage is that a permanent wave is typically formed at high pH. However, because the surface of the human skin is slightly acidic, the use of a permanent formula at high pH may cause unpleasant, irritant and negative reactions.

Thanks to being strongly reductive, thioglycolic acid is highly effective on perms, but significantly injures the hair due to the higher activity on structural ingredients of hair other than cysteine residues. Particular limitations are imparted to the use of the reducing agent on injured hair, such as bleached hair, dyed hair, etc. In addition, the application of thioglycolic acid does not guarantee a uniform perm from the root to the end of the shaft, causing the problems of splitting, cutting, or making hair brittle.

Furthermore, thioglycolic acid injures the scalp, as well. Because the health of the scalp is important for the health of the hair, it is very important not only to prevent the scalp from being injured by the permanent wave formula, but also to supply nutrition to the scalp.

### DISCLOSURE

### Technical Problem

Accordingly, the present invention has been made keeping in mind the above problems occurring in the prior art, and an object of the present invention is to provide a composition for permanent waves in which the reducing agent is adjusted to have a neutral or weak acidic pH so as to avoid irritation to the scalp and the skin of users.

It is another object of the present invention to provide a composition for permanent waves in which various amino acids are used to improve curl strength and to facilitate the invasion of the reducing agent into the cuticle layer of hair.

It is a further object of the present invention to provide a composition for permanent waves, based on naturally occurring ingredients, which is almost free of odors and skin irritation and can provide injured hair with nutrients in addition to being useful in hair treatment.

It is still a further object of the present invention to provide a composition for permanent waves in which a reducing agent almost free of skin irritation is used in combination with a citrus peel powder or extract, a green tea powder or extract, chlorella and a cactus powder or extract so as to provide the skin of the user with nutrients and to make the skin healthy.

When employed in cooperation, the citrus peel, the green tea, the chlorella and the cactus give a solution to the scalp problems associated with the reducing agent and thus make the scalp and the hair healthy in addition to aiding and improving aid and improve the function of the reducing agent.

### Technical Solution

In accordance with an aspect of the present invention, the present invention provides a composition for permanent waves, comprising: L-cysteine or a mixture of L-cysteine and L-acetylcysteine as a reducing agent, for breaking disulfide bridges of cystines to form cysteines; L-arginine for swelling keratin fibers to facilitate deep invasion of the reducing agent into the keratin fibers, said arginine being present in an amount of from 110 parts to 160 parts by weight based on 100 parts by weight of the reducing agent; and a citrus peel powder or extract, containing peptides for promoting growth of dermal papilla cells and D-limonene for preventing depilation, whereby human hair can be healthily changed from waves to straight or vice versa.

In a preferred embodiment of this aspect, L-acetylcysteine is used in an amount of 12 to 16 parts by weight based on 100 parts by weight of L-cysteine.

In another preferred embodiment, the citrus peel powder or extract is used in an amount of from 0.5 to 5 parts by weight based on 100 parts by weight of the composition.

In a further preferred embodiment, the reducing agent composed of L-cysteine alone or in combination with L-acetylcysteine is used in an amount of from 35 to 45 parts by weight based on 100 parts by weight of the composition with exclusion of aqueous solution.

Preferably, the composition may further comprises: a powder or extract of green tea leaves for exerting an astringent effect on a scalp; chlorella for exerting an anti-bacterial and deodorizing effect on the scalp; and a powder or extract of cactus for preventing a horny tissue from being generated on the scalp or desquamating upper layers of dead skin cells from the scalp, said green tea, said chlorella, and said cactus being individually used in an amount of from 0.5 to 5% by weight based on 100 parts by weight of the composition.

Preferably, the composition may further comprise L-histidine for regenerating hair.

In a preferred embodiment, the L-histidine is used in an amount of from 1.1 to 1.4 parts by weight based on 100 parts by weight of the reducing agent.

In accordance with another aspect thereof, the present invention provides a composition for permanent waves, comprising: L-cysteine or a mixture of L-cysteine and L-acetylcysteine as a reducing agent, for breaking disulfide bridges of cystines to form cysteines; L-arginine for swelling keratin fibers to facilitate deep invasion of the reducing agent into the keratin fibers, said arginine being present in an amount of from 110 parts to 160 parts by weight based on 100 parts by weight of the reducing agent; and allantoin for promoting growth of injured hair; a citrus peel powder or extract, containing peptides for promoting growth of dermal papilla cells and D-limonene for preventing depilation, whereby human hair can be changed from waves to straight or vice versa.

In a preferred embodiment of this aspect, the allantoin is used in an amount of from 0.7 to 0.9 parts by weight based on 100 parts by weight of the reducing agent.

In another preferred embodiment, the composition may further comprise keratin in an amount of from 7 to 9 parts by weight based on 100 parts by weight of the reducing agent, so as to supply the hair with nutrition.

Preferably, the composition may further comprise silk amino acids in an amount of from 0.3 to 0.4 parts by weight based on 100 parts by weight of the reducing agent so as to supply the hair with nutrition.

Preferably, the composition may further comprise an espinosilla powder or extract in an amount of from 0.1 to 0.2 parts by weight based on 100 parts by weight of the reducing agent so as to promote growth of the hair.

In a preferred embodiment, the composition may further comprise vitamin E or C in an amount of from 1 to 2 parts by weight based on 100 parts by weight of the reducing agent so as to prevent oxidation of the hair and to supply the hair with nutrition.

Preferably, the composition may further comprise guar gum or cellulose in an amount of from 0.5 to 4 parts by weight based on 100 parts by weight of the reducing agent so as to adjust viscosity of the composition.

### ADVANTAGEOUS EFFECTS

As described above, the composition for permanent waves in accordance with the present invention is expected to have the following effects.

First, composed of natural ingredient, the reducing agent can reduce the injury of both the hair and the scalp in addition to being very low in skin and scalp irritation. Also, the reducing agent can produce stable and elastic curls or straight hair within a short time with the significant reduction of a stingy odor. Further, it guarantees the maintenance of waves for a long period of time.

Second, as aids for the reducing agent, various amino acids promote the metabolism of the hair and thus make the hair glossy and smooth. Particularly, they are expected to exert very beneficial effects on injured or colored hair.

Finally, the use of extracts or powders of citrus peel, green tea, chlorella and cactus makes contribution to the health of the scalp as well as the hair, increasing the effect of the reducing agent.

### BEST MODE

A better understanding of the present invention may be obtained through the following embodiments which are set forth to illustrate, but are not to be construed as the limit of the present invention.

The main component of hair fiber is keratin which is composed of 18 amino acid residues, with the most abundant being cysteine which is responsible for 14%˜16% of them. Permanent hair shape is mainly determined by cystines formed through cysteine disulfide bridges. A permanent wave is the chemical treatment of hair hair to change straight hair into waves and vice versa. An embodiment of the present invention features the use of L-cysteine, a main component of keratin, as a reducing agent.

In this embodiment, hair is treated with cysteine to cut or break open the disulfide bridges of keratin, followed by the application of tensile force with the aid of rollers or curls to form the desired shape.

In accordance with a first embodiment of the present invention, cysteine may be used as a reducing agent alone or in combination with acetylcysteine. In the latter case, L-acetylcysteine is used as the auxiliary reducing agent. When employed as an effective ingredient of a permanent wave formula, L-acetylcysteine is characterized in that it does not crystallize even when oxidized, unlike cysteine. L-acetylcysteine, also called N-acetyl-L-cysteine, can be obtained by treating a solution of L-cysteine in aqueous sodium hydroxide with anhydrous acetic acid.

Because of being less irritant to the skin than is thioglycolic acid (although more irritating than is L-cysteine), L-acetylcysteine is used as an aid to cysteine in the present invention. When formulated with L-acetylcysteine, cysteine is expected to rarely crystallize.

In the present invention, L-acetylcysteine is used in an amount of from 10 to 20 parts by weight based on 100 parts by weight of cysteine and preferably L-acetylcysteine is used in an amount of from 12 to 16 parts by weight based on 100 parts by weight of cysteine.

In an embodiment of the present invention, thus, a mixture of cysteine and acetylcysteine at a certain ratio is used as a reducing agent with the aim of reducing the injury and irritation of both the hair and the scalp and producing stable and elastic curls or straight hair within a short time. Preferably, the reducing agent, whether it be cysteine alone or in combination with acetylcysteine, is used in an amount of from 35% to 45% by weight based on the total weight of the composition with the aqueous solution being excluded. For example, when the reducing agent is used in an amount less than 35% by weight, elastic and lasting curls and straight hair cannot be obtained although a stingy odor is reduced. On the other hand, an amount of the reducing agent greater than 45% by weight makes sure of elasticity and prolongs curls or straight hair, but injures the hair and irritates the skin and gives off a stingy odor. When combined with acetylcysteine, cysteine is preferably used in an amount of from 33% to 37% by weight based on 100 parts by weight of the composition with the exclusion of aqueous solution.

The reducing agent, whether cysteine alone or in combination with acetylcysteine, does not injure hair because it is neutral or slightly acidic, and does not give off a stingy odor because it is free of ammonia. Further, the amino acid residue(s) makes a contribution to a conditioning effect. However, cysteine, whether alone or in combination with acetylcysteine, only causes slight injury to the hair, but is weak in ability to perm, as well. Thus, the reducing agent requires extended perm time or an increased curing strength.

Located at the surface of hair, a cuticle layer is responsible for the mechanical protection of hair. It prevents the emission of moisture and the loss of proteins from hair as well as protects the hair from the invasion and injury by external matter. L-arginine can open the cuticle layer of hair to facilitate the invasion of the reducing agent into the hair.

Thus, arginine aids the rapid absorption of cysteine and acetylcysteine into hair, requiring neither the extension of perm time nor an increase of curl strength and thus makes the perm performance acceptable without injuring the hair.

Arginine, acting to facilitating the absorption of cysteine and acetylcysteine into hair, is used in an amount of from 110 parts to 160 parts by weight and preferably in an amount of from 132 parts to 135 parts by weight based on 100 parts by weight of the reducing agent.

For example, an amount of arginine less than 100 parts by weight does not guarantee good perm performance. On the other hand, when used in an amount greater than 160 parts by weight, arginine solidifies hair before the formation of a desired wave. Most preferably, arginine is used in an amount of 133 parts by weight based on 100 parts by weight of the reducing agent.

With the aim of protecting hair, keeping hair healthy, preventing hair loss and moisturizing the scalp, citrus, particularly, citrus peels are contained in the composition of the present invention. The fragrant compound “D-limonene” of citrus is responsible for the protecting activity by inhibiting 5-alpha reductase. In addition, this compound restrains moisture from being evaporated from the skin, thus moisturizing or keeping the skin, particularly the scalp, moist. Further, it is known to have therapeutic effects on skin trouble such as acne, suppuration, eczema, itching, etc. or skin disorders such as atopy, and has disinfective effects on skin injuries. The peptides and some amino acid residues obtained from the extract of citrus or citrus peel are found to promote the growth of dermal papilla cells covered by hair matrix cells by 100%.

The composition of the present invention comprises a powder or extract of green tea leaves, as well. Tannin, found in abundance in green tea, acts to bind and precipitate or shrink proteins, thus exhibiting sterilizing activity in the body. When associated with tannin, germs, made mostly of proteins, shrink and die. The more abundant tannin imparts stronger sterilization activity to green tea than red tea. Taking advantage of the sterilization and hemostatic activity thereof, tannin can inhibit the growth of demodex and bacteria on the scalp and prevent the aggravation of scalp injuries. For this reason, green tea or a green tea extract is used in the present invention.

Also, the composition of the present invention may further comprise chlorella. When eaten, chlorella tends to shift the body towards an alkaline constitution, and functions to regenerate injured cells, to protect from viral invasion, to prevent suppuration thanks to the anti-bacterial activity thereof, to remove smells from the scalp and the body thanks to the deodorization activity thereof, and to exert anti-allergic activity. Particularly, its functions of metabolism promotion, detoxification, waste removal and anti-oxidation are effective for preventing scalp aging. Further, chlorella, rich in vitamin A, makes the skin smooth and suppresses the generation of various kinds of dermatitis such as acne.

Also, the composition of the present invention may further comprise a cactus extract. It has functions of moisturizing the skin, desquamating upper layers of dead skin cells, whitening and clearing the skin tone, and preventing the scalp from being contaminated thanks to being rich in minerals and edible fibers.

In accordance with an embodiment of the present invention, the citrus peel, the green tea, the chlorella and the cactus extract are used individually in an amount of from 0.5 parts to 5 parts by weight based on 100 parts by weight of the composition.

Optionally, pine needle powder may be contained in the composition of the present invention because it has the effect of relieving itching and making hair glossy black. Particularly, it is known that a pine needle extract is pharmaceutically effective for patients with alopecia.

An extract or powder of chestnut inner shells may be used as an ingredient of the composition according to the present invention. Rich in tannin, the extract or powder of chestnut inner shells is known to astringe pores in addition to regenerating skin cells. Thus, the extract or powder may be used effectively for those who have greasy skin or wide pores.

Also, the powder of mulberry leaves and/or acorns may be employed in the composition of the present invention because these powders are effective in removing heavy metals from the scalp. Further, acorn powders can soothe burn skin.

The composition of the present invention may further comprise an extract of sweet flags. The extract is known to have various beneficial effects on hair. For example, it can deterge the scalp, make hair glossy and healthy and cause hair to grow without having negative effects on the scalp. Particularly, chlorophyll, vitamins A and C, and oxypalmitic acid are found to prevent the scalp from aging and to have astringent and anti-inflammatory activity.

An adlay powder may be used in the composition of the present invention thanks to its cosmetic activity of soothing skin trouble, invigorating the skin and preventing skin aging.

The composition of the present invention may further comprise a mung bean powder. Mung bean ingredients are found to remove sebum from the scalp, to deterge, moisturize and detoxicate the skin and to show anti-inflammatory and anti-oxidant activities. Particularly, it is effective in soothing skin troubles for sensitive skin.

Further, a licorice extract may be contained in the composition of the present invention. Usually, it is used where skin sedation, anti-inflammation and anti-allergic performance are needed. In addition, it is known to have the activity of desquamating upper layers of dead skin cells and removing sebum.

Also, ginseng may be contained in the composition of the present invention. As is well known, ginseng can invigorate, regenerate and detoxify scalp cells, and increase the elasticity of the scalp.

The composition of the present invention may further comprise an extract from Houttuyniae Herba. Having excellent antibacterial activity, Houttuyniae Herba shows an anti-inflammatory effect and cell regeneration and thus is very useful in the treatment of inflammatory scalp and atopy.

Further, the composition of the present invention may comprise black sesame since it is known to prevent aging and supply nutrition to dry skin and scalp.

Also, the present invention may further comprise tangleweed powder or an extract thereof. Tangleweed is known to have pharmaceutical effects on greasy skin and acne. Particularly, it soothes suntanned skin and supplies hair with nutrition.

The composition of the present invention may further comprise dandelion powder or dandelion extract. Rich in vitamins, saponins, sulfur ingredients, coumestrol and carotenoids, dandelion can be applied to the cleansing of the skin and the treatment of acne.

A barley powder or extract may be further contained in the composition of the present invention. It effectively facilitates blood circulation, treating skin troubles and allergies attributed to cosmetics. In addition, it promotes metabolism in the body to restrain the formation of sebum.

A powder or extract of Schizandra chinensis Baillon may be further contained in the composition of the present invention because it has the stringent activity of shrinking pores and making the skin elastic.

Paprika powder or an extract thereof may be further contained in the composition of the present invention because its abundant vitamins are useful for the treatment of atopic dermatitis.

The composition of the present invention may further comprise a powder or extract of Artemisia capillaris Thunb. Rich in capillin, pinene and vitamin, Artemisia capillaris Thunb. shows a medicinal effect on eczema. Also, vitamin C, found in abundance in the herb, is effective for hair growth.

The composition of the present invention may further comprise a black bean powder or extract. Vitamin E and unsaturated fatty acids, found in black beans, prevent hair decolorization and depilation.

A mulberry leaf powder may be further contained in the composition of the present invention because it functions to make the skin cool and is very useful in the treatment of acne.

The composition of the present invention may further comprise a powder or extract of Saururus chinensis. Saururus chinensis has an anti-inflammatory activity so that it is applied to skin regeneration and acne treatment. Its potassium salt ingredients make the skin smooth. Rutin, amino acid, saccharides, found in the herb, are also useful in the treatment of acne and dermal redness thanks to anti-inflammatory and skin regeneration effects thereof.

In accordance with an embodiment of the present invention, L-histidine, a basic amino acid, is contained in the composition so that hair regeneration is improved. This amino acid is used in an amount of 1 to 2 parts by weight and preferably in an amount of from 1.1 parts to 1.4 parts by weight based on 100 parts by weight of the reducing agent.

Various vitamins may be contained in the composition of the present invention to supply the hair with nutrition. In one embodiment, biotin is added to the composition. Biotin, also called vitamin H, is a vitamin B-complex. Serving as a cofactor in the metabolism of fatty acids and proteins, biotin helps to make hair healthy. It is also useful in the treatment of psoriasis, alopecia, dandruff, and dermatitis. Biotin is used in an amount of 2 parts to 4 parts by weight and preferably in an amount of from 2.2 parts to 2.8 parts by weight based on 100 parts by weight of the reducing agent.

Sodium citrate is used in a small amount to adjust the pH of the composition. The perm composition containing sodium citrate preferably ranges in pH from 2.0 to 10.0. When its pH is less than 2.0, the perm composition containing the pH adjustor cannot effectively fix the styled curls or straight hair. On the other hand, if the pH is over 10.0, the composition causes hair injury and skin irritation in addition to being problematic in fixing styled curl or straight hair. Sodium citrate is used in an amount of from 2 to 4 parts by weight based on 100 parts by weight of the reducing agent and preferably in an amount of from 2.2 to 2.8 parts by weight.

Finally, a viscosity-inducing component, such as polyquaternium or Hydroxyethylcellulose, may be used to adjust the viscosity of the perm composition.

Polyquaternium is natural cellulose which is artificially positively charged. It can increase the viscosity of the composition and is useful in hair treatment. Hydroxyethylcellulose is also natural cellulose extracted from plants and can be used as a substitute for polyquaternium to increase the viscosity.

In accordance with the present invention, polyquaternium or Hydroxyethylcellulose is used in an amount of from 10 parts to 15 parts by weight and preferably in an amount of from 12 parts to 13 parts by weight based on 100 parts by weight of the reducing agent.

The composition of the present invention may further comprise betaine, an amino acid-type moisturizer. It is usually used to prepare a skin lotion or cream. Because it has a glycine moiety, the betaine is often called glycine betaine. It can be also used as a substitute for polyquaternium or Hydroxyethylcellulose. Betaine is used in an amount of from 7 parts to 9 parts by weight and preferably in an amount of 8.3 parts by weight based on 100 parts by weight of the reducing agent.

Helping cellular activity either directly or indirectly, allantoin may be further used with the aim of promoting the growth of injured hair. Allantoin is present in botanical extracts of the comfrey plant and is therapeutically applied to the skin for its anti-inflammatory activity. Allantoin is safe to sensitive skin because it is allergy-free. Its activity of promoting cell proliferation and wound healing is useful in reconstructing injured hair tissue. Allantoin is used in an amount of 0.7 to 0.9 parts by weight and preferably in an amount of 0.83 parts by weight based on 100 parts by weight.

Keratin may be further contained in the composition of the present invention. As described above, keratin is the main component of hair as well as fingernails and toenails. In one embodiment of the present invention, the composition is supplemented with the main hair component keratin so as to protect hair by supplying and coating hair therewith. Keratin is formulated in an amount of from 7 to 9 parts by weight and preferably in an amount of 8.3 parts by weight based on 100 parts by weight of the reducing agent.

Silk, a natural protein fiber obtained from the cocoons of silkworms, may be further contained in the composition of the present invention. Silkworm cocoons are made of a mixture of 25:75 sericin and fibroin, which are present at the inner and the outer surface, respectively. When applied to the composition, the natural protein fibroin is responsible for supplying nutrition to hair. Silk amino acids are used in an amount of from 0.3 to 0.4 parts by weight and in an amount of 0.33 parts by weight based on 100 parts by weight of the reducing agent.

With the aim of protecting the scalp, preventing depilation and promoting hair growth, an espinosilla powder or extract may be further contained in the composition of the present invention. Espinosilla, a Mexican herb, has long been used to protect the scalp. Particularly, it is useful for dandruff prevention because it supplies nutrients and oxygen to the scalp. Espinosilla is used in an amount of from 0.10 to 0.20 parts by weight and preferably in an amount of 0.16 parts by weight based on 100 parts by weight of the reducing agent.

As an antioxidant, vitamin E may be contained in the composition of the present invention. Vitamin C may substitute for vitamin E. The antioxidant is used in an amount of from 1.0 part to 2.0 parts by weight and preferably in an amount of 1.6 parts by weight based on 100 parts by weight of the reducing agent.

As a viscosity-inducing component, guar gum or natural cellulose may be used in the composition of the present invention. Guar gum or natural cellulose is water soluble and may be used in lieu of polyquaternium or Hydroxyethylcellulose in a first embodiment and in lieu of natural betaine in a second embodiment. Guar gum or natural cellulose is used in an amount of from 0.5 parts to 4 parts by weight based on 100 parts by weight of the reducing agent.

### INDUSTRIAL APPLICABILITY

As described above, thioglycolic acid, a typical reducing agent for perm compositions, shows excellent reducing power, but suffers from the problems of injuring the hair and the scalp. In order to overcome the problem encountered in the prior art, the present invention provides a perm composition comprising L-cysteine or a mixture of L-cysteine and L-acetylcystein as a reducing agent, in combination with arginine, as a curling enhancer, for swelling keratin fibers and facilitating the reducing agent to invade deep into the keratin fibers, histidine for improving hair matrix cells and regenerating hair, and a citrus peel powder or extract for protecting the scalp, and optionally a green tea extract, chlorella, and a cactus powder or extract. The composition of the present invention protects the scalp and the skin of the users as well as making the hair healthy as a result of getting a perm.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A composition for permanent waves, comprising:
L-cysteine or a mixture of L-cysteine and L-acetylcysteine as a reducing agent, for breaking disulfide bridges of cystines to form cysteines;
L-arginine for swelling keratin fibers to facilitate deep invasion of the reducing agent into the keratin fibers, said arginine being present in an amount of from 110 parts to 160 parts by weight based on 100 parts by weight of the reducing agent; and
a citrus peel powder or extract, containing peptides for promoting growth of dermal papilla cells and D-limonene for preventing depilation,
whereby human hair can be changed from waves to straight or vice versa.

2. The composition according to claim 1, wherein L-acetylcysteine is used in an amount of 12 to 16 parts by weight based on 100 parts by weight of L-cysteine.

3. The composition according to claim 1, wherein the citrus peel powder or extract is used in an amount of from 0.5 to 5 parts by weight based on 100 parts by weight of the composition.

4. The composition according to claim 1, wherein the reducing agent composed of L-cysteine alone or in combination with L-acetylcysteine is used in an amount of from 35 to 45 parts by weight based on 100 parts by weight of the composition with exclusion of solvent.

5. The composition according to claim 1, further comprising:
a powder or extract of green tea leaves for exerting an astringent effect on a scalp;
chlorella for exerting an anti-bacterial and deodorizing effect on the scalp; and
a powder or extract of cactus for preventing a horny tissue from being generated on the scalp or desquamating upper layers of dead skin cells from the scalp, said green tea, said chlorella, and said cactus being individually used in an amount of from 0.5 to 5 parts by weight based on 100 parts by weight of the composition.

6. The composition according to claim 1, further comprising L-histidine for regenerating hair.

7. The composition according to claim 6, wherein L-histidine is used in an amount of from 1.1 to 1.4 parts by weight based on 100 parts by weight of the reducing agent.

8. A composition for permanent waves, comprising:
L-cysteine or a mixture of L-cysteine and L-acetylcysteine as a reducing agent, for breaking disulfide bridges of cystines to form cysteines;
L-arginine for swelling keratin fibers to facilitate deep invasion of the reducing agent into the keratin fibers, said arginine being present in an amount of from 110 parts to 160 parts by weight based on 100 parts by weight of the reducing agent;
allantoin for promoting growth of injured hair; and
a citrus peel powder or extract, containing peptides for promoting growth of dermal papilla cells and D-limonene for preventing depilation,
whereby human hair can be changed from waves to straight or vice versa.

9. The composition according to claim 8, wherein the allantoin is used in an amount of from 0.7 to 0.9 parts by weight based on 100 parts by weight of the reducing agent.

10. The composition according to claim 8, further comprising keratin in an amount of from 7 to 9 parts by weight based on 100 parts by weight of the reducing agent, so as to supply the hair with nutrition.

11. The composition according to claim 8, further comprising silk amino acids in an amount of from 0.3 to 0.4 parts by weight based on 100 parts by weight of the reducing agent so as to supply the hair with nutrition.

12. The composition according to claim 8, further comprising an espinosilla powder or extract in an amount of from 0.1 to 0.2 parts by weight based on 100 parts by weight of the reducing agent so as to promote growth of the hair.

13. The composition according to claim 8, further comprising vitamin E or C in an amount of from 1 to 2 parts by weight based on 100 parts by weight of the reducing agent so as to prevent oxidation of the hair and to supply the hair with nutrition.

14. The composition according to claim 8, further comprising guar gum or cellulose in an amount of from 0.5 to 4 parts by weight based on 100 parts by weight of the reducing agent so as to adjust viscosity of the composition.
